(19) United States
(12) Patent Application Publication
(10) Pub. No.: US 2023/0255967 A1
(43) Pub. Date: Aug. 17, 2023

(54) PHARMACEUTICAL COMPOSITIONS COMPRISING A DIAMINOPYRIMIDINE DERIVATIVE OR PHARMACEUTICALLY ACCEPTABLE SALT THEREOF AND PROCESSES FOR PREPARING THE SAME

(71) Applicant: YUHAN CORPORATION, Seoul (KR)

(72) Inventors: Dong-Min Park, Gyeonggi-do (KR); Su-Hyeon Kim, Gyeonggi-do (KR); Eun-Pa Cheon, Gyeonggi-do (KR); Tae-Lin Ha, Gyeonggi-do (KR); Jun-Mo Yang, Gyeonggi-do (KR); Yoong-Sik Park, Gyeonggi-do (KR)

(73) Assignee: YUHAN CORPORATION, Seoul (KR)

(21) Appl. No.: 18/014,262

(22) PCT Filed: Jul. 1, 2021
(86) PCT No.: PCT/KR2021/008327

§ 371 Date: Jan. 3, 2023

(30) Foreign Application Priority Data
Jul. 9, 2020 (KR) 10-2020-0084595

(51) Int. Cl. A61K 31/506; A61K 9/20

(52) U.S. Cl. CPC A61K 31/506; A61K 9/2013; A61K 9/2095; A61K 9/2054; A61K 9/2027

(57) ABSTRACT

The present invention provides a pharmaceutical composition comprising (S)—N-(1-(2-((4-amino-3-nitrophenyl)amino)-6-propylpyrimidin-4-yl)pyrrolidin-3-yl)acetamide or a pharmaceutically acceptable salt thereof having an activity as a 5-HT₄ receptor agonist and an acidifying agent; and a process for preparing the same.

### TECHNICAL FIELD

The present invention relates to a pharmaceutical composition comprising a diaminopyrimidine derivative or pharmaceutically acceptable salt thereof having an activity as a 5-HT₄ receptor agonist and a process for preparing the same.

### BACKGROUND ART

The diaminopyrimidine derivative of Formula 1 below has a chemical name of (S)—N-(1-(2-((4-amino-3-nitrophenyl)amino)-6-propylpyrimidin-4-yl)pyrrolidin-3-yl) a cetamide. The diaminopyrimidine derivative of Formula 1 or pharmaceutically acceptable salt thereof (e.g., hydrochloride) functions as a 5-HT₄ receptor agonist, and therefore can be usefully applied for preventing or treating dysfunction in gastrointestinal motility, one of the gastrointestinal diseases, such as gastroesophageal reflux disease (GERD), constipation, irritable bowel syndrome (IBS), dyspepsia, post-operative ileus, delayed gastric emptying, gastroparesis, intestinal pseudo-obstruction, drug-induced delayed transit, or diabetic gastric atony (WO 2012/115480).

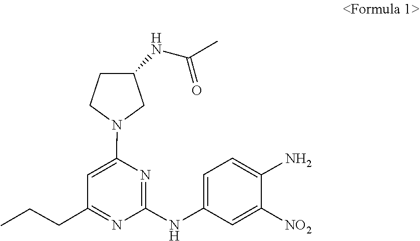 [SMILES: CCCc1cc(N2CC[C@H](NC(C)=O)C2)nc(Nc2ccc(N)c([N+](=O)[O-])c2)n1]

WO 2019/221522 discloses an improved process for preparing the diaminopyrimidine derivative of Formula 1 or salt thereof, along with novel crystalline forms and processes for preparing the same.

When the diaminopyrimidine derivative of Formula 1 or pharmaceutically acceptable salt thereof is formulated into a composition for oral administration, it may be considered to formulate into the form of an immediate-release (IR) pharmaceutical composition, which has an absorption mechanism that the active ingredient is immediately-released in the stomach and then delivered to the small intestine. For for-mulating into such an immediate-release pharmaceutical composition, it is required to minimize the effect of changes in pH in the stomach, for example associated with food or co-administered drugs (e.g., antacids, etc.). The average pH in the stomach in fed state ranges from pH 3 to pH 5 (i.e., not constant) and may show a higher pH depending on the individual. Co-administration of drugs such as antacids also increases the pH in the stomach. When a drug affected by the pH environment in the stomach is formulated according to a conventional formulation method, variations in drug release may occur and thus the absorption rate and bioavailability may change.

### DISCLOSURE OF INVENTION

### Technical Problem

The present inventors have found that the diaminopyrimidine derivative of Formula 1 or pharmaceutically acceptable salt thereof exhibits a pH-dependent physicochemical property (e.g., dissolution rate), thereby being greatly affected by a change in pH in the stomach. The present inventors have also found that, when carrying out the formulation process thereof using an acidifying agent, the acidifying agent provides a low pH microenvironment in the stomach during the release of the diaminopyrimidine derivative of Formula 1 or a pharmaceutically acceptable salt thereof (e.g., the HCl salt thereof), which makes it possible to minimize the effect of changes in the pH environment in the stomach and thus to formulate into an immediate-release pharmaceutical composition that can provide not only rapid drug release but also rapid and high gastrointestinal absorption rate in various gastric pH environments.

Therefore, it is an object of the present invention to provide a pharmaceutical composition (e.g., an immediate-release pharmaceutical composition) comprising a diaminopyrimidine derivative of Formula 1 or pharmaceutically acceptable salt thereof and an acidifying agent.

It is another object of the present invention to provide a process for preparing said pharmaceutical composition.

### Solution to Problem

In accordance with an aspect of the present invention, there is provided a pharmaceutical composition comprising a compound of Formula 1 or pharmaceutically acceptable salt thereof; and an acidifying agent.

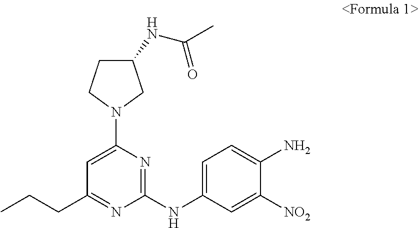 [SMILES: CCCc1cc(N2CC[C@H](NC(C)=O)C2)nc(Nc2ccc(N)c([N+](=O)[O-])c2)n1]

In the pharmaceutical composition of the present invention, the pharmaceutically acceptable salt of the compound of Formula 1 may be an acid addition salt, preferably a HCl salt, of the compound of Formula 1.

The acidifying agent may be selected from the group consisting of citric acid, edetic acid, malic acid, and a mixture thereof, preferably citric acid. The acidifying agent may be present preferably in an amount ranging from 0.1 to 10 parts by weight, based on 1 part by weight of the compound of Formula 1 or pharmaceutically acceptable salt thereof. In an embodiment, the acidifying agent is present in an amount of about 1 part by weight, based on 1 part by weight of the compound of Formula 1 or pharmaceutically acceptable salt thereof.

The pharmaceutical composition of the present invention may further comprise one or more excipients selected from the group consisting of an additive, a disintegrant, a lubricant, and a binder. The pharmaceutical composition of the present invention may be an immediate-release pharmaceutical composition; and be in the form of an oral solid dosage form selected from the group consisting of a powder, a granule, a pellet, a tablet, and a capsule.

In accordance with another aspect of the present invention, there is provided a process for preparing a pharmaceutical composition in the form of a tablet, the process of which comprises compressing a mixture of the compound of Formula 1 or pharmaceutically acceptable salt thereof, an acidifying agent, and a pharmaceutically acceptable excipient. The pharmaceutically acceptable excipient may be one or more selected from the group consisting of an additive, a disintegrant, a binder, and a lubricant.

In accordance with still another aspect of the present invention, there is provided a process for preparing a pharmaceutical composition in the form of a tablet, the process of which comprises (a) preparing granules comprising the compound of Formula 1 or pharmaceutically acceptable salt thereof; and an acidifying agent, and (b) compressing a mixture of the granules prepared in the step (a) and a pharmaceutically acceptable excipient.

In the process for preparing a pharmaceutical composition of the present invention in the form of a tablet, the granules in the step (a) may further comprise an additive. In an embodiment, the step (a) may be carried out by granulating through spraying an aqueous solution comprising the compound of Formula 1 or pharmaceutically acceptable salt thereof and an acidifying agent onto an additive fluidizing in a fluid bed granulator. In said embodiment, the additive may be microcrystalline cellulose. In the process for preparing a pharmaceutical composition of the present invention in the form of a tablet, the pharmaceutically acceptable excipient in the step (b) may be one or more selected from the group consisting of an additive, a disintegrant, a binder, and a lubricant.

### Advantageous Effects of Invention

It has been found by the present invention that the diaminopyrimidine derivative of Formula 1 or pharmaceutically acceptable salt thereof exhibits a pH-dependent physicochemical property (e.g., dissolution rate), thereby being greatly affected by a change in pH in the stomach. It has been also found by the present invention that, when carrying out the formulation process thereof into immediate-release pharmaceutical composition using an acidifying agent (preferably citric acid), it is possible to minimize the effect of changes in the pH environment in the stomach on the diaminopyrimidine derivative of Formula 1 or a pharmaceutically acceptable salt thereof (e.g., the HCl salt thereof). That is, the immediate-release pharmaceutical composition of the present invention can provide not only rapid drug release but also rapid and high gastrointestinal absorption rate in various gastric pH environments.

### BRIEF DESCRIPTION OF DRAWINGS

FIG. 1 shows the results obtained by measuring the dissolution rates at pH 1.2 of the immediate-release tablets prepared in Example 1-1 and Comparative Example.

FIG. 2 shows the results obtained by measuring the dissolution rates at pH 4.0 of the immediate-release tablets prepared in Example 1-1 and Comparative Example.

FIG. 3 shows the results obtained by measuring the dissolution rates at pH 6.8 of the immediate-release tablets prepared in Example 1-1 and Comparative Example.

### BEST MODE FOR CARRYING OUT THE INVENTION

The present invention provides a pharmaceutical composition comprising a compound of Formula 1 or pharmaceutically acceptable salt thereof; and an acidifying agent.

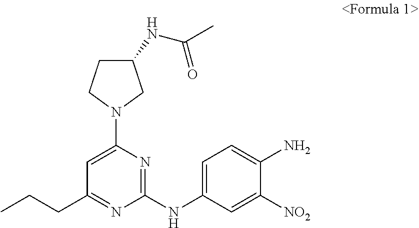 [SMILES: CCCc1cc(N2CC[C@H](NC(C)=O)C2)nc(Nc2ccc(N)c([N+](=O)[O-])c2)n1]

In the pharmaceutical composition of the present invention, the pharmaceutically acceptable salt of the compound of Formula 1 may be in forms of various salts disclosed in WO 2012/115480, for example inorganic salts, organic salts or metal salts. The inorganic salts include hydrochloride, phosphate, sulfate, bisulfate, and the like. The organic acid salts include malate, maleate, citrate, fumarate, besylate, camsylate, edicylate and the like. The metal salts include calcium salt, sodium salt, magnesium salt, strontium salt, potassium salt and the like. The pharmaceutically acceptable salt of the compound of Formula 1 may be preferably an acid addition salt, more preferably a HCl salt (i.e., hydrochloride), of the compound of Formula 1. The pharmaceutical composition of the present invention may comprise the compound of Formula 1 or pharmaceutically acceptable salt thereof in therapeutically effective amounts, for example, ranging from 0.03 to 20 mg, preferably from 0.05 to 10 mg, per unit formulation (per unit pharmaceutical composition), but is not limited thereto.

It has been found by the present invention that, when carrying out the formulation process using an acidifying agent, it possible to minimize the effect of changes in the pH environment in the stomach on the diaminopyrimidine derivative of Formula 1 or pharmaceutically acceptable salt thereof (e.g., the HCl salt thereof) exhibiting a pH-dependent physicochemical property (e.g., dissolution rate). The acidifying agent can maintain a high dissolution rate of the diaminopyrimidine derivative of Formula 1 or pharmaceutically acceptable salt thereof even in a high pH environment such as pH 6.8, thereby ensuring not only rapid drug release but also rapid and high absorption rate in various pH environments.

The acidifying agent may be selected from the group consisting of citric acid, edetic acid, malic acid, and a mixture thereof, preferably citric acid. The acidifying agent may be present in an amount of 10 parts by weight or less, preferably in an amount ranging from 0.1 to 10 parts by weight, more preferably in an amount ranging from 0.5 to 2 parts by weight, based on 1 part by weight of the compound of Formula 1 or pharmaceutically acceptable salt thereof. In an embodiment, the acidifying agent is present in an amount of about 1 part by weight, based on 1 part by weight of the compound of Formula 1 or pharmaceutically acceptable salt thereof. When the amount of the acidifying agent exceeds 10 parts by weight based on 1 part by weight of the compound of Formula 1 or pharmaceutically acceptable salt thereof, the stability of the obtained pharmaceutical composition may be deteriorated. In addition, when the amount of the acidifying agent is less than 0.1 part by weight based on 1 part by weight of the compound of Formula 1 or pharmaceutically acceptable salt thereof, it may be difficult to minimize the effect of changes in the pH environment in the stomach.

The pharmaceutical composition of the present invention may further comprise excipients conventionally used in an immediate-release formulation, for example one or more excipients selected from the group consisting of an additive, a disintegrant, a lubricant, and a binder, in addition to the compound of Formula 1 or pharmaceutically acceptable salt thereof and said acidifying agent. The additive (or diluent) includes lactose, microcrystalline cellulose, mannitol, and a mixture thereof, but is not limited thereto. The disintegrant includes corn starch, crospovidone, croscarmellose sodium, sodium starch glycolate, low-substituted hydroxypropyl cellulose, and a mixture thereof, but is not limited thereto. The binder includes povidone, hydroxypropyl cellulose, hydroxypropyl methylcellulose, and a mixture thereof, but is not limited thereto. The lubricant includes silicon dioxide, talc, stearic acid, magnesium stearate, sodium stearyl fumarate, and a mixture thereof, but is not limited thereto. In an embodiment, the pharmaceutical composition of the present invention may further comprise microcrystalline cellulose as an additive (or diluent), crospovidone as a disintegrant, and a mixture of silicon dioxide and sodium stearyl fumarate as a lubricant. Said excipients may be used in amounts conventionally used in an immediate-release formulation, and the amounts thereof are not particularly limited.

The pharmaceutical composition of the present invention may be e.g., an immediate-release pharmaceutical composition. The dosage forms thereof are not particularly limited. For example, the pharmaceutical composition of the present invention may be in the form of an oral solid dosage form selected from the group consisting of a powder, a granule, a pellet, a tablet, and a capsule, preferably in the form of a tablet.

The present invention includes, within its scope, a process for preparing the pharmaceutical composition described above. That is, the present invention includes, within its scope, a process for preparing an immediate-release pharmaceutical composition selected from the group consisting of a powder, a granule, a pellet, a tablet and a capsule. For example, the pharmaceutical composition of the present invention in the form of a powder or a capsule may be prepared by mixing the active ingredient (the compound of Formula 1 or pharmaceutically acceptable salt thereof), an acidifying agent, and a pharmaceutically acceptable excipient or by filling the resulting mixture into capsules. The pharmaceutical composition of the present invention in the form of a granule or a pellet may be prepared by together or separately granulating or pelletizing the active ingredient (the compound of Formula 1 or pharmaceutically acceptable salt thereof) and an acidifying agent, along with a pharmaceutically acceptable excipient.

For example, the pharmaceutical composition of the present invention in the form of a tablet may be prepared according to a direct compression method or an indirect compression method. In the process for preparing the pharmaceutical composition of the present invention in the form of a tablet, the pharmaceutically acceptable salt of the compound of Formula 1 and the acidifying agent are same as described above with respect to the pharmaceutical composition of the present invention.

The process according to a direct compression method may comprise compressing a mixture of the compound of Formula 1 or pharmaceutically acceptable salt thereof, an acidifying agent, and a pharmaceutically acceptable excipient. The pharmaceutically acceptable excipient may be one or more selected from the group consisting of an additive, a disintegrant, a binder, and a lubricant. The mixture may be obtained by mixing all components simultaneously; or by mixing some of the components, and then additionally mixing the other components. Those skilled in the art will be able to prepare said mixture according to methods conventionally practiced in the field of pharmaceutics, based on the disclosures of the present description.

The process according to an indirect compression method may comprise (a) preparing granules comprising the compound of Formula 1 or pharmaceutically acceptable salt thereof; and an acidifying agent, and (b) compressing a mixture of the granules prepared in the step (a) and a pharmaceutically acceptable excipient.

In the process according to an indirect compression method, the granules in the step (a) may further comprise an additive. The granulating of the step (a) may be carried out according to a dry granulation method or a wet granulation method. For example, the wet granulation process may be carried out by preparing a binder solution in which an acidifying agent is dissolved or dispersed and then kneading the active ingredient and a pharmaceutically acceptable excipient with the binder solution. It has been found by the present invention that the granulation process can be carried out without using a binder (i.e., using only water), through spraying an aqueous solution comprising the compound of Formula 1 or pharmaceutically acceptable salt and an acidifying agent onto an additive (or diluent) fluidizing in a fluid bed granulator. Therefore, in an embodiment, the step (a) may be carried out by granulating through spraying an aqueous solution comprising the compound of Formula 1 or pharmaceutically acceptable salt thereof and an acidifying agent onto an additive fluidizing in a fluid bed granulator. In said embodiment, the additive (or diluent) may be microcrystalline cellulose.

In the process for preparing a tablet according to an indirect compression method, the pharmaceutically acceptable excipient in the step (b) may be one or more selected from the group consisting of an additive, a disintegrant, a binder, and a lubricant. When the step (a) is carried out using a fluid bed granulator, the additive (or diluent) among the excipients used in the step (b) may be the same as or different from the additive (or diluent) used in the step (a). The weight ratio of the additives (or diluents) used in the step (a) and the step (b) may 2 to 5: 1, preferably 3 to 4: 1, but the weight ratio thereof may vary depending on the kinds of the additives (or diluents) used.

The present invention will be described in further detail with reference to the following examples and experimental examples. These examples and experimental examples are for illustrative purposes only and are not intended to limit the scope of the present invention.

In the following examples and experimental examples, the “Compound 1” refers to (S)—N-(1-(2-((4-amino-3-nitrophenyl)amino)-6-propylpyrimidin-4-yl)pyrrolidin-3-yl) a cetamide hydrochloride.

### Example 1: Preparation of Tablets Containing an Acidifying Agent

Immediate-release tablets comprising Compound 1 were prepared according to the components and amounts shown in Table 1. The amounts of Table 1 represent the weight (mg) of each component per unit tablet. Specifically, for preparing the tablets of Example 1-1 to 1-3, Compound 1 and the acidifying agent were dissolved in purified water (in the ratio of about 150 ml per 1 mg of Compound 1). For preparing the tablet of Comparative Example, Compound 1 was dissolved in purified water (in the ratio of about 150 ml per 1 mg of Compound). While fluidizing microcrystalline cellulose in a fluid bed granulator (Glatt, USA), each granulation was carried out by spraying the aqueous solution prepared above thereon (inlet: 60-70° C., product: 35-40° C., exhaust: 30-40° C.). The resulting granules were mixed with microcrystalline cellulose, crospovidone, and silicon dioxide, and then additionally mixed with sodium stearyl fumarate. The resulting mixtures were compressed using a tablet press machine (XP-1, Korsch), respectively, to prepare the immediate-release tablets.

TABLE 1

| | | Example (mg) | | | Comparative |
|---|---|---|---|---|---|
| | Component | 1-1 | 1-2 | 1-3 | Example (mg) |
| Intra- | Compound 1 | 3 | 3 | 3 | 3 |
| granules | Microcrystalline | 59 | 59 | 59 | 62 |
| | cellulose | | | | |
| | Citric acid | 3 | — | — | — |
| | Edetic acid | — | 3 | — | — |
| | malic acid | — | — | 3 | — |
| Extra- | Microcrystalline | 18 | 18 | 18 | 18 |
| granules | cellulose | | | | |
| | Crospovidone | 5 | 5 | 5 | 5 |
| | Silicon dioxide | 1 | 1 | 1 | 1 |
| | Sodium stearyl | 1 | 1 | 1 | 1 |
| | fumarate | | | | |

| | | | | |
|---|---|---|---|---|
| Total weight | 90 | 90 | 90 | 90 |

### Example 2: Preparation of Tablets Containing Citric Acid as an Acidifying Agent

Immediate-release tablets comprising Compound 1 were prepared in the same procedures as in Example 1-1, according to the components and amounts shown in Table 2. The amounts of Table 2 represent the weight (mg) of each component per unit tablet.

TABLE 2

| | | Example (mg) | | | | | |
|---|---|---|---|---|---|---|---|
| | Component | 2-1 | 2-2 | 2-3 | 2-4 | 2-5 | 2-6 |
| Intra- | Compound 1 | 0.05 | 0.1 | 0.25 | 0.3 | 0.5 | 1 |
| granules | Microcrystalline | 63.95 | 63.9 | 63.75 | 63.7 | 63.5 | 63 |
| | cellulose | | | | | | |
| | Citric acid | 1 | 1 | 1 | 1 | 1 | 1 |
| Extra- | Microcrystalline | 18 | 18 | 18 | 18 | 18 | 18 |
| granules | cellulose | | | | | | |
| | Crospovidone | 5 | 5 | 5 | 5 | 5 | 5 |
| | Silicon dioxide | 1 | 1 | 1 | 1 | 1 | 1 |
| | Sodium stearyl | 1 | 1 | 1 | 1 | 1 | 1 |
| | fumarate | | | | | | |

| | | | | | | |
|---|---|---|---|---|---|---|
| Total weight | 90 | 90 | 90 | 90 | 90 | 90 |

Experimental Example 1: Dissolution Test

Dissolution tests on the immediate-release tablets prepared in Example 1-1 and Comparative Example were carried out at pH 1.2, pH 4.0, and pH 6.8 according to the Apparatus 1 (Basket Apparatus) of U.S. Pharmacopeia. The dissolution medium was prepared according to the buffer composition described in the US Pharmacopoeia. Samples were taken from each dissolution medium at predetermined times and the amount of Compound 1 in each sample was measured by Ultra High Performance Liquid Chromatography (UPLC) under the following conditions.

<UPLC Conditions>
Detector: UV-Vis Spectrophotometer
Column: ZORBAX Eclipse Plus C18 Rapid Resolution HD (2.1×50 mm, 1.8 μm)
Flow rate: 0.2 mL/min
Injection volume: 5 μL
Mobile phase:

Mobile phase A: Ammonium bicarbonate buffer (pH 7.0)

Mobile phase B: Mixed solution of acetonitrile and methanol (4/1, v/v)

| Time (minute) | Mobile phase A (%) | Mobile phase B (%) |
|---|---|---|
| 0.0 | 80 | 20 |
| 0.6 | 80 | 20 |
| 3.5 | 10 | 90 |
| 4.0 | 10 | 90 |
| 4.1 | 80 | 20 |
| 6.0 | 80 | 20 |

Temperature: about 40° C.
Wavelength: 272 nm

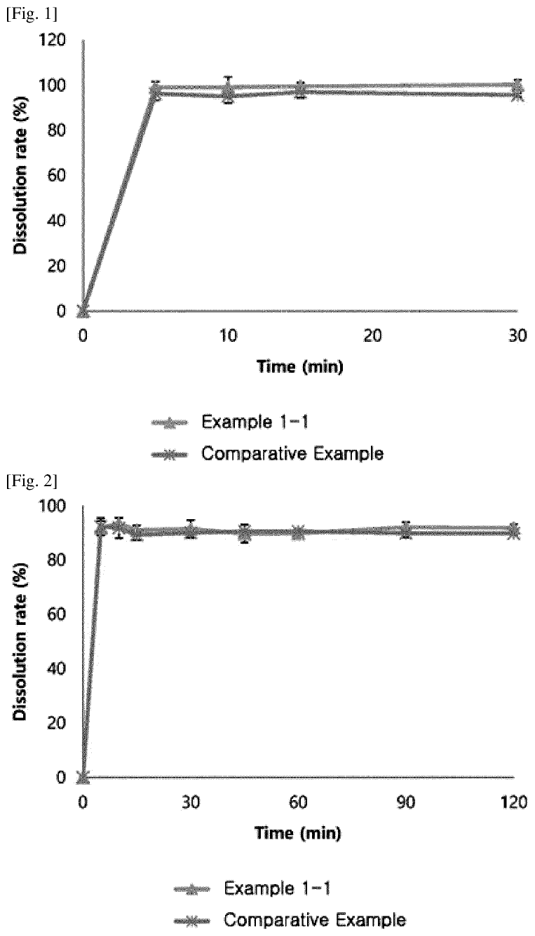

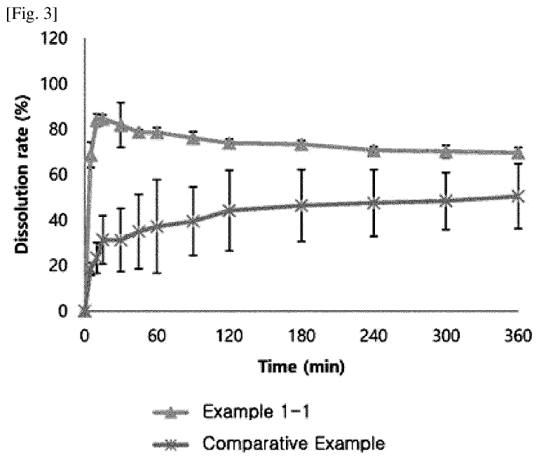

The results obtained by carrying out the dissolution test as described above are shown in FIGS. 1 to 3. As can be seen from the results of FIGS. 1 to 3, the tablet obtained according to the present invention (i.e., the tablet of Example 1-1) showed a high dissolution rate from the beginning, regardless of the pH of the dissolution medium. On the other hand, the tablet of Comparative Example showed a remarkably low dissolution rate (including the initial dissolution rate) under the condition of pH 6.8. Therefore, the immediate-release tablet obtained according to the present invention can ensure not only rapid drug release but also rapid and high gastrointestinal absorption rate in various gastric pH environments.

Experimental Example 2: Stability Test

Stability tests under the stress condition were carried out by placing the immediate-release tablets prepared in Examples 2-1 to 2-6 in an HDPE container (an high density polyethylene bottle) and then storing for 24 months under the conditions of a temperature of 25±2° C. and a relative humidity of 60±5%. Each sample stored for 24 months was dissolved in a test solution (i.e., a solution obtained by mixing 1000 mL of water, 1000 mL of methanol and 2 mL of trifluoroacetic acid), and then the amount of total degradation products was determined by Ultra High Performance Liquid Chromatography (UPLC) under the following conditions.

<UPLC Conditions>
Detector: UV-Vis Spectrophotometer
Column: ACQUITY UPLC® HSS T3 (2.1×100 mm, 1.8 μm)
Flow rate: 0.3 mL/min
Injection volume: 5 μL
Mobile phase:

Mobile phase A: Ammonium bicarbonate buffer (pH 7.0)

Mobile phase B: Mixed solution of acetonitrile and methanol (4/1, v/v)

| Time (minute) | Mobile phase A (%) | Mobile phase B (%) |
|---|---|---|
| 0.0 | 95 | 5 |
| 2.0 | 95 | 5 |
| 8.5 | 50 | 50 |
| 11.0 | 48 | 52 |
| 15.0 | 10 | 90 |
| 17.0 | 10 | 90 |
| 17.1 | 95 | 5 |
| 20.0 | 95 | 5 |

Temperature: about 40° C.
Wavelength: 272 nm

The results of the stability test as described above are shown in the following Table 3.

TABLE 3

| | Amount of total degradation products |
|---|---|
| Example 2-1 | 4.68% |
| Example 2-2 | 1.38% |
| Example 2-3 | 1.13% |
| Example 2-4 | 0.96% |
| Example 2-5 | 0.82% |
| Example 2-6 | 0.69% |

As can be seen from the results of Table 3, as the ratio of the acidifying agent increased, the amount of degradation products tends to increase. From the above results, it can be seen that the acidifying agent may be used in an amount of 10 parts by weight or less, preferably 0.1 to 10 parts by weight, more preferably 0.5 to 2 parts by weight, and particularly preferably about 1 part by weight, based on 1 part by weight of Compound 1.

1. A pharmaceutical composition comprising a compound of Formula 1 or pharmaceutically acceptable salt thereof; and an acidifying agent.

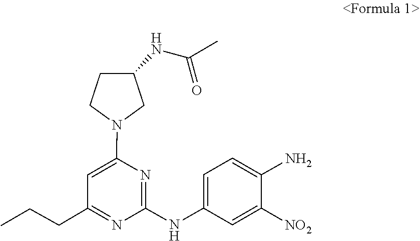 [SMILES: CCCc1cc(N2CC[C@H](NC(C)=O)C2)nc(Nc2ccc(N)c([N+](=O)[O-])c2)n1]

2. The pharmaceutical composition according to claim 1, wherein the pharmaceutically acceptable salt of the compound of Formula 1 is an acid addition salt of the compound of Formula 1.

3. The pharmaceutical composition according to claim 2, wherein the pharmaceutically acceptable salt of the compound of Formula 1 is a HCl salt of the compound of Formula 1.

4. The pharmaceutical composition according to claim 1, wherein the acidifying agent is selected from the group consisting of citric acid, edetic acid, malic acid, and a mixture thereof.

5. The pharmaceutical composition according to claim 4, wherein the acidifying agent is citric acid.

6. The pharmaceutical composition according to claim 1, wherein the acidifying agent is present in an amount ranging from 0.1 to 10 parts by weight, based on 1 part by weight of the compound of Formula 1 or pharmaceutically acceptable salt thereof.

7. The pharmaceutical composition according to claim 6, wherein the acidifying agent is present in an amount of about 1 part by weight, based on 1 part by weight of the compound of Formula 1 or pharmaceutically acceptable salt thereof.

8. The pharmaceutical composition according to claim 1, further comprising one or more excipients selected from the group consisting of an additive, a disintegrant, a lubricant, and a binder.

9. The pharmaceutical composition according to claim 1, wherein the pharmaceutical composition is an immediate-release pharmaceutical composition.

10. The pharmaceutical composition according to claim 1, wherein the pharmaceutical composition is in the form of an oral solid dosage form selected from the group consisting of a powder, a granule, a pellet, a tablet, and a capsule.

11. A process for preparing a pharmaceutical composition in the form of a tablet, the process of which comprises compressing a mixture of a compound of Formula 1 or pharmaceutically acceptable salt thereof, an acidifying agent, and a pharmaceutically acceptable excipient.

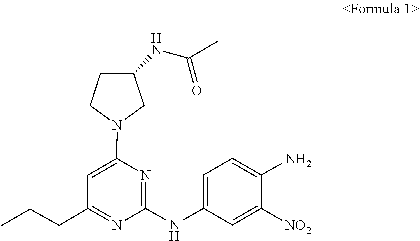 [SMILES: CCCc1cc(N2CC[C@H](NC(C)=O)C2)nc(Nc2ccc(N)c([N+](=O)[O-])c2)n1]

12. A process for preparing a pharmaceutical composition in the form of a tablet, the process of which comprises:
(a) preparing granules comprising a compound of Formula 1 or pharmaceutically acceptable salt thereof; and an acidifying agent, and 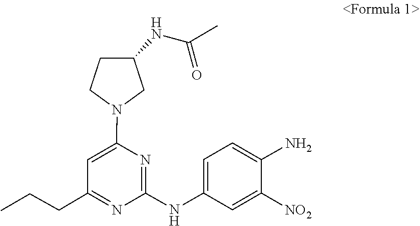 [SMILES: CCCc1cc(N2CC[C@H](NC(C)=O)C2)nc(Nc2ccc(N)c([N+](=O)[O-])c2)n1]
(b) compressing a mixture of the granules prepared in the step (a) and a pharmaceutically acceptable excipient.

13. The process according to claim 11, wherein the pharmaceutically acceptable salt of the compound of Formula 1 is an acid addition salt of the compound of Formula 1.

14. The process according to claim 13, wherein the pharmaceutically acceptable salt of the compound of Formula 1 is a HCl salt of the compound of Formula 1.

15. The process according to claim 11, wherein the acidifying agent is selected from the group consisting of citric acid, edetic acid, malic acid, and a mixture thereof.

16. The process according to claim 15, wherein the acidifying agent is citric acid.

17. The process according to claim 11, wherein the acidifying agent is used in an amount ranging from 0.1 to 10 parts by weight, based on 1 part by weight of the compound of Formula 1 or pharmaceutically acceptable salt thereof.

18. The process according to claim 17, wherein the acidifying agent is used in an amount of about 1 part by weight, based on 1 part by weight of the compound of Formula 1 or pharmaceutically acceptable salt thereof.

19. The process according to claim 12, wherein the granules in the step (a) further comprise an additive.

20. The process according to claim 12, wherein the step (a) is carried out by granulating through spraying an aqueous solution comprising the compound of Formula 1 or pharmaceutically acceptable salt thereof and an acidifying agent onto an additive fluidizing in a fluid bed granulator.

21. The process according to claim 19, wherein the additive is microcrystalline cellulose.

22. The process according to claim 11, wherein the pharmaceutically acceptable excipient is one or more selected from the group consisting of an additive, a disintegrant, a binder, and a lubricant.
